(19) United States
(12) Patent Application Publication
(10) Pub. No.: US 2016/0007900 A1
(43) Pub. Date: Jan. 14, 2016

(54) Point-of-Care Device for Monitoring Renal Function

(71) Applicant: THE REGENTS OF THE UNIVERSITY OF CALIFORNIA, Oakland, CA (US)

(72) Inventors: Jeffrey Veale, Los Angeles, CA (US); Fang Wei, North Hills, CA (US)

(21) Appl. No.: 14/770,613

(22) PCT Filed: Feb. 28, 2014
(86) PCT No.: PCT/US14/19594

§ 371 Date: Aug. 26, 2015

Related U.S. Application Data
(60) Provisional application No. 61/771,377, filed on Mar. 1, 2013.

(51) Int. Cl. A61B 5/20; G01N 27/327; A61B 5/145

(52) U.S. Cl. CPC A61B 5/201; A61B 5/14546; G01N 27/3275

(57) ABSTRACT

The present invention relates to a rapid and accurate polymer-based electrochemical point-of-care (POC) single platform assay for a multi-biomarker detection from whole blood to monitor renal function, including the identification of allograft dysfunction.

### CROSS-REFERENCE TO RELATED APPLICATIONS

This application is a U.S. national phase application filed under 35 U.S.C. §371 claiming benefit to International Patent Application No. PCT/US2014/019594, filed Feb. 28, 2014, which in turn is entitled to priority under 35 U.S.C. §119(e) to U.S. Provisional Patent Application Ser. No. 61/771,377 filed Mar. 1, 2013, the entire disclosure of which is incorporated by reference herein in its entirety.

### STATEMENT REGARDING FEDERALLY SPONSORED RESEARCH OR DEVELOPMENT

This invention was made with government support under Grant No. TR000124, awarded by the National Institutes of Health (NIH). The government has certain rights in the invention.

### BACKGROUND OF THE INVENTION

Kidney transplant recipients who have abnormally high creatinine levels in their blood often have allograft dysfunction secondary to rejection. Creatinine has become the preferred marker for renal dysfunction and is readily available in hospital clinical settings.

Electrochemical (EC) sensors, especially bioaffinity sensors, such as DNA hybridization biosensors or immunosensors, have gained considerable attention in recent years and have been extensively used in clinical diagnostic laboratory settings for biosensing and detection (Wei et al., 2009, Pediatr Res 67:458-468; Wei et al., 2009, Clin. Cancer Res. 15:4446-4452; Wei et al., 2008, Nucleic Acids Res 36:e65; Bange et al., 2005, Biosens Bioelectron 20:2488-2503; Chaubey et al., 2002, Biosens Bioelectron 17:441-456; Wilson et al., 1992, Biosens Bioelectron 7:165-185; Walcarius, 2001, Chem Mater 13:3351-3372; Ivnitski et al., 1999, Biosens Bioelectron 14:599-624). Such devices exploit selective binding of specific target species by surface-confined receptor molecules for triggering informative electrical signals. Electrochemical affinity biosensors commonly rely on specific antibody-, DNA-, or aptamer-recognition events. Bioelectronic assays with enzyme tracers have been extensively applied, because of the signal amplification from biocatalytic reactions, and hold promise for the ultrasensitive detection of nucleic acids and/or proteins. In addition, EC sensors have achieved high sensitivity and specificity and are simple, with respect to sample processing and instrument operation. EC sensors have the potential to be transformed from a laboratory-based instrument to a point-of-care (POC) device. Electrochemical biosensors have thus become a very promising area of research and development in clinical diagnostic testing (Wei et al., 2008, Nucleic Acids Res 36:e65; Subrahmanyam et al., 2001, Biosens Bioelectron 16:631-637; Soldatkin et al., 2002, Talanta 58:351-357).

Because of the biocompatibility, convenience in fabrication, and low cost, conducting polymer-based biosensors provide ideal platforms for various applications of POC devices (Subrahmanyam et al., 2001, Biosens Bioelectron 16:631-637; Lakshmi, et al., 2006, Talanta 70:272-280; Madaras and Buck, 1996, Anal Chem 68:3832-3839; Li et al., 2012, Anal Chim Acta 711:83-90). A conducting polymer (CP) has been widely applied as an easy-to-fabricate and biocompatible substrate material for a diverse array of analytes (Cosnier, 1999, Biosens Bioelectron 14:443-456; Cosnier, 2005, Electroanalysis 17:1701-1715; Cosnier, 2007, Anal Lett 40:1260-1279; Fan et al., 2003, Proc Natl Acad Sci USA 100:6297-6301; Ramanaviciene and Ramanavicius, 2002, Crit Rev Anal Chem 32:245-252). Most of the CP-based biosensors immobilize probes, such as an oligonucleotide or antibody and enzyme, directly onto polymer film by simply mixing them with the monomer before the electropolymerization (Wei et al., 2009, Small 5:1784-1790; Gerard et al., 2002, Biosens Bioelectron 17:345-359). In this rapid and simple procedure, the embedding of the CP matrix with the desired molecule without any labeling is very important for probe immobilization. Especially for the detection of small molecules (e.g., chemical, hormone, drugs, etc.) based on affinity, modifications are required to generate the binding site between the molecule and the surface. By applying the copolymerization with polymer matrix, the molecule can be directly embedded into the surface without additional modification. The total reaction time is from several seconds to minutes, at room temperature, with a regular biocompatible buffer.

Diagnostic methods for detecting renal dysfunction of kidney transplant recipients has not changed in over 20 years. For example, the approach involves patients coming to the hospital clinic periodically for their blood to be drawn to determine the creatinine level. Although the gold standard measurements for renal dysfunction are radiolabeled ¹²⁵I-iodothalamate and inulin, these tests are difficult to perform and generally are unavailable (Baracskay et al., 1997, Clin Nephrol 47:222-228). Therefore, creatinine has become the preferred marker for renal dysfunction and readily available in essentially all hospital clinical settings. Creatinine is a byproduct of muscle metabolism and typically remains in a steady state balanced out by renal elimination. Kidney transplant recipients who have an abnormally high creatinine level in their blood often have allograft dysfunction secondary to rejection. The recipient is then scheduled for an ultrasound-guided biopsy of the allograft to confirm tissue diagnosis. If the rejection is caught early, it can be easily reversed with current immunosuppressants. However, over time, transplant recipients have their creatinine measured infrequently as they grow tired of the inconvenience of enduring traffic and waiting room lines for their blood to be drawn. Therefore, by the time rejection is detected, it is often symptomatic, and irreversible damage has been done to the allograft. In fact, the deceased donor waiting list is burdened by 17% of candidates who have lost their previous graft to rejection and are awaiting their second, third, or fourth transplantation.

The traditional method for detecting creatinine is based on a modified Jaffe reaction, which is widely used in both laboratory and clinical detection. The Jaffe reaction is based on the orange-red color produced by creatinine reacting with alkaline picrate. The sensitivity of the Jaffe reaction is 0.5 mg/dL of creatinine in samples. The entire detection time is 30 min with a sample volume of at least 1 mL. However, the specificity of the Jaffe reaction is limited. Several interferents in clinical samples will affect the signal readout, such as metabolites and drugs (e.g., glucose, proteins, ketones, hemoglobin, bilirubin, pyruvic acid, etc.) and cephalosporines (e.g., cefoxitin, cephalotin, cefatril, cefazolin, etc.). In addition, this detection requires expensive spectrometer and sample pretreatment, which limit the application to monitor creatinine over the long run.

There are also several promising techniques developed for monitoring creatinine in blood, including enzymatic catalysis of creatinine (direct and indirect) and antibody-based affinity detection. The enzymatic catalysis of creatine usually has the enzyme for creatinine (creatinine iminohydrolase (Chou et al., 2009, IEEE Sens J 9:665-672), creatinine amidohydrolase, creatine amidinohydrolase, and creatinine deiminase (Lad et al., 2008, Anal Chem 80:7910-7917)). Specifically for the creatinine electrochemical sensor, the existing detections are mostly based on the electrochemical catalysis of creatinine. In this detection, creatinine amidohydrolase catalyzes the hydrolytic reaction converting creatinine to creatine, followed by the sarcosine oxidase (SOX) reaction with the mediator system (Madaras and Buck, 1996, Anal Chem 68:3832-3839). The amperometric current is measured simultaneously with a 60-μL untreated blood sample within 90 s. However, this reaction also has a low specificity from the interference of creatine in the samples (Benkert et al., 2000, Anal Chem 72:916-921; Straseski et al., 2011, Clin Chem 57:1566-1573). This harbors concerns that discrepant results may affect clinical management. These discrepancies may be related to elevations in hematocrit interfering with the enzymatic method.

Antibody-based affinity detection is more specific for creatine measurements in complex body fluids. The reaction is usually based on competitive reaction, which measures the signal decrease with the existence of creatinine. Pioneering investigations have been done in this area. Usually, a modification of creatine as the capture molecule, or an optical detector, is needed, which limits the application as a POC device (Lo and Tsai, 1994, Clin Chem 40:2326-2327; Benkert et al., 2000, Electroanalysis 12:1318-1321). In addition, multiplexing measurements are not applicable for this technology.

It is also worth noting that these traditional methods can typically only measure single biomarkers. However, recent research shows that combinational biomarkers, provide more accurate information for renal function diagnostics. The recent finding published in JAMA (Peralta et al., 2011, JAMA 305(15):1545-52) that Creatinine combined with Cystatin-C improves the accuracy of predicting renal function, yet unfortunately there is no system or device to advance this finding as a diagnostic.

Development of a point-of-care testing (POCT) device to specifically measure blood creatinine levels would allow patients the convenience of monitoring their allograft function frequently in the comfort of their own home. Besides the obvious improvement in quality of life, a creatinine POCT device would likely detect rejection at an earlier stage, when it is more easily addressed and reversed. Ultimately, this device could lessen the burden of patients that return to the waiting list by extending graft survival.

In short, traditional diagnostic methods are burdened in low efficiency, long waiting-room times, and the inconvenience of patients traveling to hospital clinics. The ability to monitor and determine the allograft dysfunction using a convenient and accurate POC device would revolutionize the field. Thus, there is a need in the art for a rapid and accurate assay for creatinine detection from whole blood using a POCT device based on creatinine-specific immunoassay. The present invention satisfies this need.

### SUMMARY OF THE INVENTION

A device for monitoring renal function in a subject is described. The device includes an array of units on a substrate, each unit comprising an electrode chip including a working electrode, a counter electrode, and a reference electrode, wherein the working electrode of at least one unit is coated with a conducting polymer embedded or functionalized with a first marker of renal function.

In one embodiment, the working electrode, counter electrode, and reference electrode are comprised of a conductive material. In another embodiment, the first marker of renal function is creatinine. In another embodiment, the current in the electrode chip is altered when the first marker of renal function binds to an antibody directed against the first marker. In another embodiment, the antibody is conjugated to horseradish-peroxidase (HRP). In another embodiment, the conducting polymer comprises pyrrole. In another embodiment, the conducting polymer is electropolymerized on the working electrode by applying a cyclic square-wave electric field to the device. In another embodiment, the working electrode of at least one unit is coated with a conducting polymer embedded with an antibody directed against a second marker of renal function. In another embodiment, the current in the electrode chip is altered when the antibody directed against the second marker binds to the second marker. In another embodiment, the second marker of renal function is cystatin-C.

A method of monitoring renal function in a subject is also described. The method includes the steps of obtaining a blood sample from the subject, mixing a first portion of the sample with a solution comprising an antibody directed against a first marker of renal function to form a mixture, adding the mixture to a first electrode chip on a device, the electrode chip comprising a working electrode, a counter electrode, and a reference electrode, wherein the working electrode is coated with a conducting polymer embedded with the first marker of renal function, and measuring the current in the electrode chip, wherein the magnitude of a change in current, compared to the current measured when a mixture comprising the antibody but not comprising the sample is added to the electrode chip, is correlated to the quantity of the first marker in the sample.

In one embodiment, the first marker of renal function is creatinine. In another embodiment, the method further includes applying a cyclic square-wave electric field to the electrode chip during the adding step. In another embodiment, the method further includes mixing a second portion of the sample with a solution comprising an antibody directed against a second marker of renal function to form a second mixture, adding the second mixture to a second electrode chip on the device, the second electrode chip comprising a working electrode, a counter electrode, and a reference electrode, wherein the working electrode is coated with a conducting polymer embedded with an antibody directed against the second marker of renal function, measuring the current in the second electrode chip, wherein the magnitude of the current is correlated to the quantity of the second marker in the sample. In another embodiment, the second marker of renal function is cystatin-C.

The present invention also includes a system for monitoring renal function in a subject. The system includes an electrochemical sensor chip having at least one well, wherein the at least one well contains a working electrode coated with a conducting polymer functionalized with at least one capture molecule, and at least one antibody that specifically binds to a biomarker of renal function. The at least one antibody is mixed with a blood sample from the subject and added to the at least one well, such that when an electric current is applied to the sample in the at least one well, at least some the biomarker binds to the capture molecule, thereby creating a measurable change in electric current in the sample that is indicative of the presence of the biomarker in the sample.

In one embodiment, the biomarker is creatinine. In another embodiment, the capture molecule is creatinine. In another embodiment, the system further includes a first antibody that specifically binds to creatinine and a second antibody that specifically binds to cystatin-C, and at least two wells in the electrochemical sensor chip, wherein the capture molecule in the first well specifically binds to creatinine and the capture molecule in the second well specifically binds to cystatin-C. In another embodiment, the change in current in the sample is measurable within 5 minutes after the sample has been loaded into the well.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an illustration of the direct measurement of creatinine from a blood sample via a conducting polymer electrochemical sensor. The reaction is based on the amperometric measurement of the HRP-conjugated creatinine antibody bound with creatinine.

FIG. 2 is an illustration of an electrochemical sensor array for the creatinine measurements and the calibration curves. (A) 16-array bare gold sensor chip with a counter electrode (CE), a working electrode (WE), and a reference electrode (RE). (B) Cyclic square-wave electric potential applied during the polymerization and the reaction. For EC polymerization, the low voltage was +350 mV and the high voltage was +950 mV. For the surface recognition, the low voltage was −200 mV and the high voltage was +300 mV. (C) Amperometric curves of the spiked creatine into blood sample. Current signal for calibration is the difference between the sample signal and the blank control signal. (D) Sensitivity of the EC sensor for creatinine detection of blood samples with linear fit (R²=0.98). The linear fitting equation is ΔI (nA)=98.98+19.17×creatinine concentration (mg/dL). Mean value and standard deviation are both illustrated with triplet experiments.

FIG. 3 is an illustration of a data correlation between the EC sensor and the Ronald Reagan UCLA Medical Center traditional Jaffe-based assay on paired clinical samples. The linear correlation is illustrated (R²=0.94).

FIG. 4 is an illustration of the multiplexing detection of creatinine and cystatin-C on the same electrochemical sensor.

FIG. 5 is an illustration of calibration curves for multiplexing measurements in PBS buffer.

FIG. 6 is an illustration of calibration curves of the assay in pooled blood samples.

FIG. 7 is an illustration of the robustness study of the assay.

FIG. 8 is an illustration of a correlation between the assay of the invention and the CTRL central lab.

FIG. 9 is an illustration of the differentiation between Rejection, Stable and Healthy Controls.

FIG. 10 is an illustration of the dynamic process of monitoring patients transitioning through rejection.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical point-of-care devices. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

“Instructional material,” as that term is used herein, includes a publication, a recording, a diagram, or any other medium of expression which can be used to communicate the usefulness of the sensor, reader, chip, nucleic acid, peptide, antibody and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a sample. The instructional material of the kit may, for example, be affixed to a container that contains the sensor, reader, chip, nucleic acid, peptide, antibody and/or compound of the invention or be shipped together with a container that contains such items. Alternatively, the instructional material may be shipped separately from the container with the intention that the recipient uses the instructional material and the kit components cooperatively.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

As used herein, “Affinity moiety” refers to a binding molecule, such as an antibody, aptamer, peptide or nucleic acid, that specifically binds to a particular target molecule, such as an analyte, biomarker or other targeted molecule to be detected in a testing sample.

By the term “specifically bind” or “specifically binds,” as used herein, is meant that a first molecule preferentially binds to a second molecule, but does not necessarily bind only to that second molecule. As used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “detection reagent” refers to an agent comprising an affinity moiety that specifically binds to an analyte, biomarker or other targeted molecule to be detected in a sample. Detection reagents may include, for example, a detectable moiety, such as a radioisotope, a fluorescent label, a magnetic label, and enzyme, or a chemical moiety such as biotin or digoxigenin. The detectable moiety can be detected directly, or indirectly, by the use of a labeled specific binding partner of the detectable moiety. Alternatively, the specific binding partner of the detectable moiety can be coupled to an enzymatic system that produces a detectable product. Further, the detectable moiety may be detected via a change in electrical current.

The term “antibody,” as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

As used herein, an “immunoassay” refers to any binding assay that uses an antibody capable of binding specifically to a target molecule to detect and quantify the target molecule.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The present invention relates to a rapid and accurate polymer-based electrochemical POC single platform assay for a multi-biomarker detection from whole blood to identify allograft dysfunction. While the present invention is described generally for the testing of a whole blood sample, it should be appreciated that partial or processed blood samples may be used, or even other tissue or sample types, such as plasma, serum and urine, provided such alternative sample types carry the targeted markers to be analyzed. Non-limiting example of such markers include creatinine and cystatin-C or any other markers associated with renal function, such as blood urea nitrogen (BUN). It should be appreciated that any number of biomarkers can be integrated to the assay platform, including, without limitation, 4, 8, 16, 32 or 64 biomarkers per array.

The noninvasive detection of graft dysfunction in transplant recipients via the present invention enables clinicians to reverse damaging processes in the early stages of rejection when these processes are more easily reversible. An example of this would be to detect graft dysfunction secondary to rejection. As a result, and increase in immunosuppressive medications such as pulsing steroids may be administered, thereby reversing the rejection process before significant damage occurs in the transplanted organ. As contemplated herein, the present invention includes a multiplexing electrochemical sensor for combinational renal function biomarkers. The device utilizes a small sample volume with high accuracy. In addition, multiple markers are measured simultaneously on the device with single sample loading. The present invention is also suitable for patient monitoring, which would allow patients the convenience of monitoring their renal function frequently in the comfort of their own home, or as a POC assay in a clinic setting. Besides the obvious positive impact on quality-of-life, the present invention may further detect graft dysfunction at an earlier stage when it is more easily reversed. Accordingly, the device may significantly reduce the cost to the health care system, by decreasing the burden of patients returning to clinics and laboratories. Ultimately, the present invention provides a platform that improves overall graft survival rates, which necessarily lessens the number of patients returning to the wait-list for subsequent renal transplantations.

In one embodiment, the present invention is based on the antibody affinity between a capture molecule and target marker, which is specific compared to the traditional enzymatic systems. As contemplated herein, the assay platform may be organized as any type of affinity binding assay or immunoassay as would be understood by those skilled in the art, including competitive binding assays, sandwich assays, and the like. Enzymatic systems suffer from inaccuracy as they rely on chemical reactions that have interference with molecules commonly found in body fluids (e.g., glucose, fructose, ketones, antibiotics, etc.). In another embodiment, the present invention includes a single platform for multiple renal function biomarker measurements, instead of a single marker. Currently, there is no such technology or device available for this purpose. In another embodiment, the present invention creates tremendous efficiencies in that it is simple, rapid and robust. For example, only small sample volumes are needed (e.g., 10 μl) and less than 15 minutes run time are needed. A user may perform the test by a single finger-prick at their convenience, according to the protocol provided. The blood droplet would then be measured with the device within minutes. Multiple marker levels would be provided by the device. By providing statistical analysis the user would have an estimate of their risk, and by utilizing available networking systems, the results can be quickly transmitted for review by a clinician for further assessment.

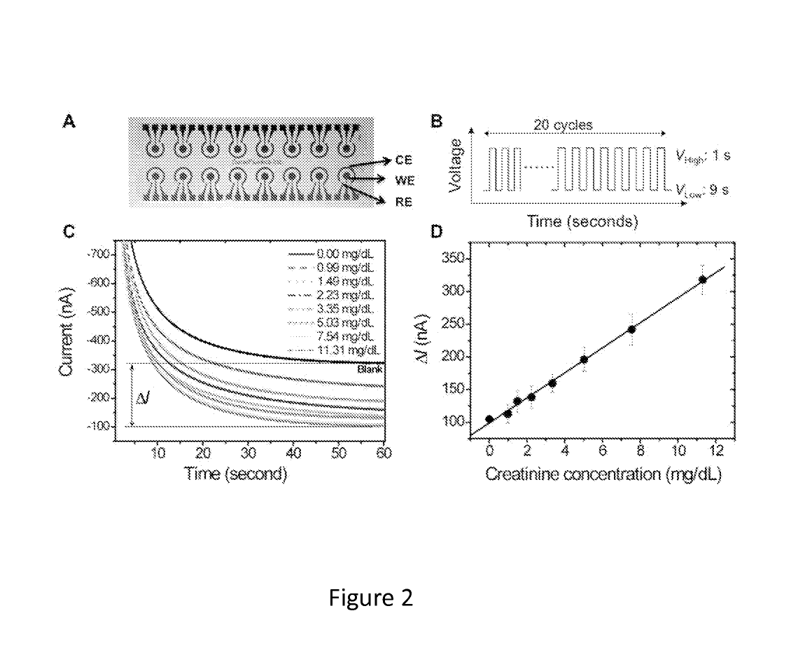

In one embodiment, the EC sensor is an array of electrode chips (GeneFluidics, USA). Each unit of the array has a working electrode, a counter electrode, and a reference electrode. The three electrodes may be constructed of bare gold or other conductive material before the reaction, such that the specimens may be immobilized on the working electrode (see FIG. 2A). Electrochemical current can be measured between the working electrode and counter electrode under the potential between the working electrode and the reference electrode. The potential profile could be a constant value, a linear sweep, or a cyclic square wave, for example. An array of plastic wells may be used to separate each three-electrode set, which helps avoid the cross contamination between different sensors. A conducting polymer may also be deposited on the working electrodes as a supporting film, and in some embodiments, as a surface to functionalize the working electrode with a capture moiety, such as an antibody used in a sandwich assay, or the target biomarker or mimetic in a competitive assay. As contemplated herein, any conductive polymer may be used, such as polypyrroles, polanilines, polyacetylenes, polyphenylenevinylenes, polythiophenes and the like.

In a preferred embodiment, a cyclic square wave electric field is generated across the electrode within the sample well. The positive potential in the csw E-field helps the molecules accumulate onto the working electrode, while the negative potential removes the weak nonspecific binding, to generate enhanced specificity. Further, the flapping between positive and negative potential across the cyclic square wave also provides superior mixing during incubation, without disruption of the desired specific binding, which accelerates the binding process and results in a faster test or assay time. In one embodiment, a square wave cycle may consist of a longer low voltage period and a shorter high voltage period, to enhance binding partner hybridization within the sample. While there is no limitation to the actual time periods selected, examples include 5 to 60 second low voltage periods and 0.5 to 3 second high voltage periods. In a preferred embodiment, each square-wave cycle consists of 9 s at low voltage and 1 s at high voltage. For hybridization, the low voltage range may be between −0.8 to +0.4V and the high voltage range may between 0 to +1.0V. In a preferred embodiment, the low voltage may be around −200 mV and the high voltage may be around +300 mV. In some embodiments, the total number of square wave cycles may be between 2-50. In a preferred embodiment, 20 cyclic square-waves are applied for each surface reaction. With the csw E-field, both the hybridization and protein binding are finished on the same chip within minutes, while, previously, these processes must be completed separately and the incubation time varies from 1 h to 24 h. In some embodiments, the total detection time from sample loading is less than 30 minutes. In other embodiments, the total detection time from sample loading is less than 20 minutes. In other embodiments, the total detection time from sample loading is less than 10 minutes. In other embodiments, the total detection time from sample loading is less than 5 minutes.

A multi-channel EC reader (GeneFluidics) controls the electrical field applied onto the array sensors and reports the amperometric current simultaneously. In practice, solutions can be loaded onto the entire area of the three-electrode region including the working, counter, and reference electrodes, which are confined and separated by the array of plastic wells. After each step, the EC sensors can be rinsed with ultrapure water or other washing solution and then dried, such as under pure N₂. In some embodiments, the sensors are single use, disposable sensors. In other embodiment, the sensors are reusable.

Antibodies embedded in the conductive polymer or otherwise used to functionalize the working electrode surface, or those antibodies used to bind the targeted biomarker within the subject sample may be constructed according to any protocol known in the art for the generation of antibodies. Similarly, any known label or detection moiety may be associated with such antibodies. In one non-limiting example, the antibody is a sheep polyclonal antibody against creatinine (Abnova, USA), where the immunogen is creatinine conjugated with BSA, and horseradish peroxidase (HRP) is conjugated to the antibody with a NH₂-biotin-label kit, following the enclosed instruction from the company (Dojindo, USA). There is no limitation to the concentrations of such antibodies used, and may be optimized as needed by the user. In one example, the final concentration of a HRP-anticreatinine antibody is 250 μg/mL.

Due to the enhanced sensitivity of the present invention, very small volumes may be used to perform the desired assays. For example, the sample size of whole blood from the subject may be between 5-100 microliters. In a preferred embodiment, the sample size need only be about 40 microliters. There is no limitation to the actual or final sample size to be tested. In certain embodiments, only 1-100 microliters of 250 μg/mL antibody is needed for mixing with the subject's sample. In a preferred embodiment, only ten microliters (10 μL) of HRP-conjugated anticreatinine antibody is needed for mixing with 40 μL of raw blood sample solution at room temperature.

In one embodiment, the assay is a competitive assay for measuring creatinine in the sample. In this embodiment, creatinine is embedded into the conductive polymer layer atop the working electrode within the device well, and a HRP-anticreatinine antibody is mixed with a subject's sample, such that the antibody binds to creatinine within the sample. The sample is then applied to the well and a cyclic square wave electric field is generated through the desired cycle number. The decreased current of the HRP-antibody is proportional to the level of creatinine in the samples, as shown in FIG. 2C, where the electrochemical signal is the current generated by the redox cycles between TMB, the HRP reporter enzyme, and H₂O₂ (see Experimental Example 1, below). The concentration of the creatinine can be calculated according to its specific calibration curve, where calibration curves are obtained for each individual sample. Alternatively, a single universal calibration curve may be generated that simplifies the process for clinical application.

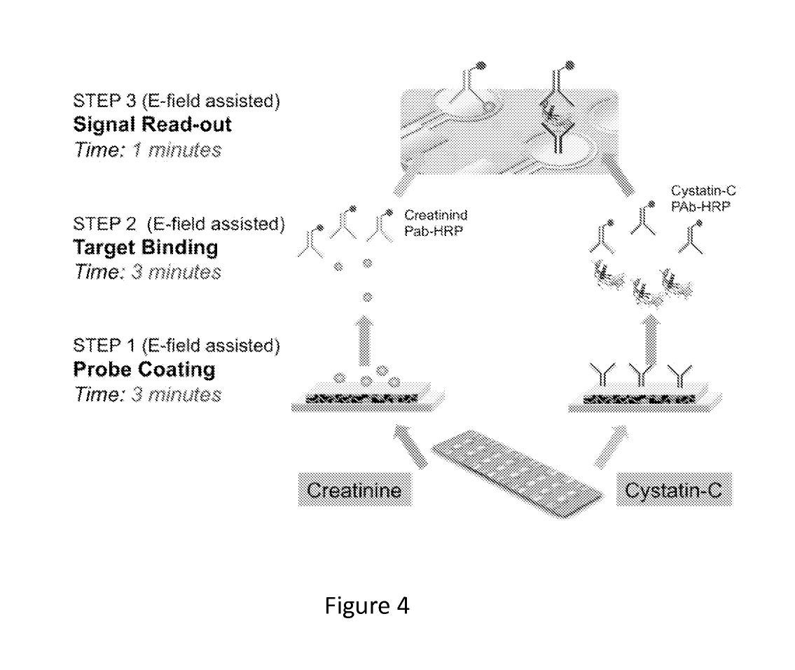

In another embodiment, the assay is a sandwich assay for measuring cystatin-C in the sample, either separately or simultaneously with the measurement of creatinine. In this embodiment and as generally illustrated in FIG. 4, an anticystatin-C antibody is embedded into the conductive polymer layer atop a working electrode within the device well, and a HRP-anticystatin-C antibody is mixed with a subject's sample, such that the antibody binds to cystatin-C within the sample. The sample is then applied to the well and a cyclic square wave electric field is generated through the desired cycle number.

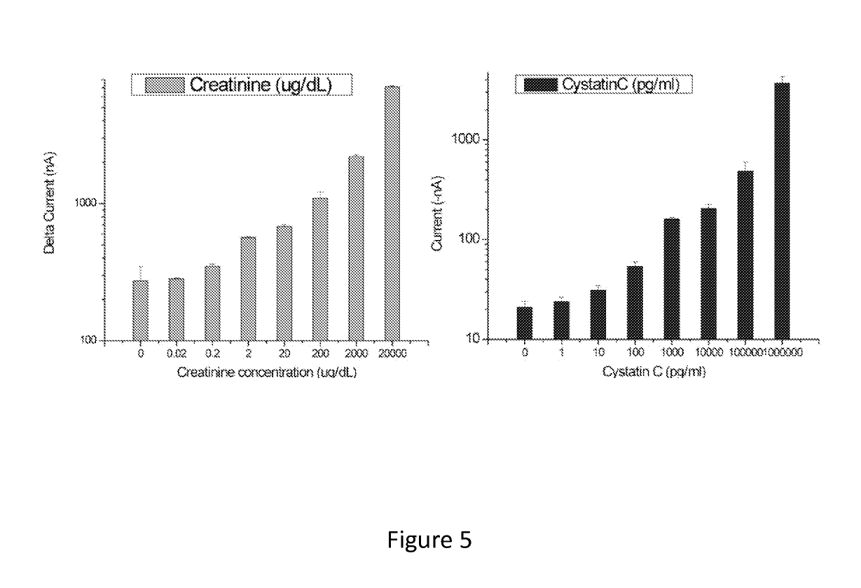

In certain embodiments, creatinine can be detected at 0.46 mg/dL, which is the ideal clinical range of 0 mg/dL to 11.3 mg/dL for detecting graft dysfunction. In a buffer system, creatinine can be detected at 0.2 ug/dL, and 1 pg/ml for cystatin-C. The dynamic range is 20 mg/dl to 0.2 ug/dl for creatinine and 1 ug/ml to 1 pg/ml for cystatin-C, as shown in FIG. 5.

As described herein, the present invention provides a platform for detecting the presence or quantity of at least one biomarker in a sample. In particular, the present invention provides a point of care testing platform for detecting the presence of, or amount of, creatinine and/or cystatin-C in a sample taken from a subject to be tested, as these markers are indicators of renal function in the subject.

Accordingly, the present invention relates to a method of monitoring renal function in a subject. In one embodiment, the method may be performed as a competitive assay and includes the steps of obtaining a sample from the subject, adding an antibody labeled with a detectable moiety directed against a targeted marker of renal function to the sample, applying the sample to an electrode chip coated with a conducting polymer previously embedded or functionalized with the targeted marker of renal function, and measuring the current in the electrode chip. The magnitude of the decrease in current in the sample is correlated to the quantity of the marker in the sample, as compared to the current measured when a control sample of the antibody is added to the electrode chip. In another embodiment, the method may be performed as a sandwich type assay and includes the steps of obtaining a sample from the subject, embedding or fixing a capture antibody or other molecule directed against a targeted marker of renal function to a conductive polymer coating on an electrode chip, adding an antibody labeled with a detectable moiety directed against a targeted marker of renal function to the sample being tested, applying the sample to the electrode chip coated with the capture antibody, and measuring either the detectable moiety or alternatively the magnitude of the current in the sample.

The noninvasive detection of graft dysfunction in transplant recipients via the present invention enables clinicians to reverse damaging processes in the early stages of rejection when these processes are more easily reversible. An example of this would be to detect graft dysfunction secondary to rejection. As a result, and increase in immunosuppressive medications such as pulsing steroids may be administered, thereby reversing the rejection process before significant damage occurs in the transplanted organ. Ultimately, the present invention provides a platform that improves overall graft survival rates, which necessarily lessens the number of patients returning to the wait-list for subsequent renal transplantations.

In alternative embodiments, the present invention may further be suitable for use with radiology and/or oncology specialties that rely on renal function before implementing contrast or chemotherapy regimens. Further, application of the present invention may extend outside of medical practice, such as to determine dehydration status of military personnel in the field.

The present invention further includes an assay kit containing the EC sensor array and instructions for the set-up, performance, monitoring, and interpretation of the assays of the present invention. Optionally, the kit may include reagents for the detection of at least one of the biomarkers. The kit may also optionally include the sensor reader.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out embodiments of the present invention, and are not to be construed as limiting in any way the remainder of the disclosure.

### Example 1

### Creatine Detection from Whole Blood

### Sample Collection

Freshly collected whole blood samples from renal transplant recipients were obtained under the auspices of an ongoing UCLA IRB for immune monitoring. Patient specimens were assigned a numeric code to remove any identification. Nineteen (19) blood samples were utilized for this study. All samples were run in triplicate to assess experimental precision and human error variance.

### Traditional Laboratory Assay for Creatinine Comparison Measurement

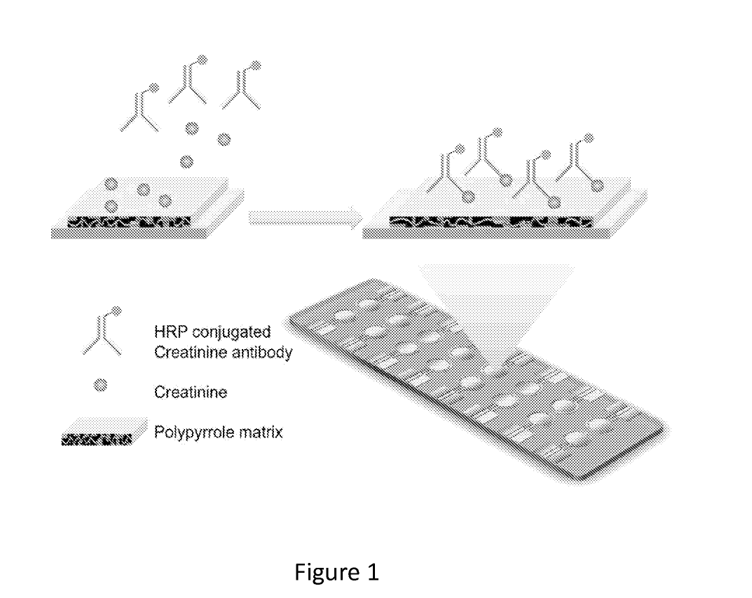

For this diagnostic validation, the creatinine levels were measured via a modified Jaffe reaction. Creatinine in the blood samples reacted with alkaline picrate and generated orange-red color products. The signal readouts were based on the spectra from the orange-read color products. All automated creatinine tests had been run on the Olympus 5400 (Olympus Diagnostic Systems) calorimetric assay. This was conducted by the routine diagnostic flow at the UCLA Ronald Reagan Medical Center Central Laboratory. The routine turnaround time for detection at the central laboratory was 4 h and required 5 mL of blood sample. The electrochemical sensor is based on the competitive amperometric measurement from the creatinine in the samples (see FIG. 1). (Wei et al., 2009, Clin. Cancer Res. 15:4446-4452; Wei et al., 2009, Small 5:1784-1790)

### EC Sensors and Reader

The EC sensor is an array of 16 bare gold electrode chips (GeneFluidics, USA). Each unit of the array has a working electrode, a counter electrode, and a reference electrode. The three electrodes are bare gold before the reaction, and the specimens are immobilized on the working electrode (see FIG. 2A). Electrochemical current is measured between the working electrode and counter electrode under the potential between the working electrode and the reference electrode. The potential profile could be a constant value, a linear sweep, or a cyclic square wave. An array of 16 plastic wells separate each three-electrode set, which avoids the cross contamination between different sensors. A conducting polymer was deposited on the working electrodes as the supporting film. The 16-channel EC reader (GeneFluidics) controls the electrical field applied onto the 16 array sensors and reports the amperometric current simultaneously. All the electrical potentials in the following steps are referred to the gold reference electrode, which was determined to be +218 mV vs SCE by measuring cyclic voltammetric curves of 0.1 mM [Fe(CN)₆]^{3-/4-}. For the experiments, solutions were loaded onto the entire area of the three-electrode region including the working, counter, and reference electrodes, which were confined and separated by the array of 16 plastic wells. After each step, the EC sensors were rinsed with ultrapure water (18.3 MΩ cm) and then dried under pure N₂.

### Conducting Polymer Sensor Fabrication for Creatinine

A cyclic square-wave electrical field (csw E-field) was applied for the electropolymerization and surface-recognition processes, which provide a more effective and versatile way to control the assay (see FIG. 2B). With the csw E-field, both the hybridization and protein binding are finished on the same chip within minutes, while, previously, these processes must be completed separately and the incubation time varies from 1 h to 24 h. The positive potential in the csw E-field helps the molecules accumulate onto the working electrode, while the negative potential removes the weak nonspecific binding, which generates high specificity. The flapping between positive and negative potential also provides good mixing during the incubation, which accelerates the binding process as well. Each square-wave cycle consisted of 9 s at low voltage and 1 s at high voltage. For EC polymerization, the low voltage was +350 mV and the high voltage was +950 mV. For the surface recognition, the low voltage was −200 mV and the high voltage was +300 mV. In total, 20 cyclic square-waves were applied for each surface reaction, which lasted 200 s.

The 16-array gold electrochemical sensor is first coated with creatinine embedded in the polypyrrole-conducting polymer. For electropolymerization, the 20 mg/dL creatinine (Abnova, USA) was diluted together with pyrrole (Sigma, USA) in 1×PBS (pH 7.5, Invitrogen, USA) in a volume ratio of 1:50. Potassium chloride was added at a final concentration of 300 mM to achieve high ionic strength. The final concentration of pyrrole was 10 mM. After loading of the mixture onto the gold electrode, a csw E-field was applied for electropolymerization. Each square-wave consisted of 9 s at a potential of +350 mV and 1 s at +950 mV, and 20 square-wave cycles were applied. The entire process lasted 200 s. After the polymerization, the electrode was rinsed with ultrapure water (18.3 MΩ cm) and then dried under pure N₂.

### Amperometric Creatinine Measurement

The final concentration is reported based on the individual calibration curve for each sample. The antibody is a sheep polyclonal antibody against creatinine (Abnova, USA). The immunogen is creatinine conjugated with BSA. Horseradish peroxidase (HRP) was conjugated to the antibody with the NH₂-biotin-label kit, following the enclosed instruction from the company (Dojindo, USA). The final concentration of the HRP-anticreatinine antibody is 250 μg/mL.

Ten microliters (10 μL) of HRP-conjugated anticreatinine antibody was first mixed with 40 μL of raw blood sample solution at room temperature. The mixture then was transferred onto the electrodes for competitive reaction between the creatinine in solution and on the polymer matrix. Twenty (20) cyclic square-waves that consisted of 9 s at a potential of −300 mV and 1 s at +200 mV were applied. The entire process lasted 200 s. After that, the sensor was washed with water and then dried under pure N₂. The amperometric measurements then were carried out in the presence of 3,3-,5,5-tetramethylbenzidine (TMB/H₂O₂, Neogen Corp., USA) low-activity substrate at −200 mV. The decreased current of the HRP-antibody is proportional to the level of creatinine in the samples (FIG. 2C). In the experiments, the electrochemical signal was the current generated by the redox cycles between TMB, the HRP reporter enzyme, and H₂O₂. All experiments were performed at room temperature.

For each individual sample, a calibration curve was obtained as well as the signal from the sample. In the calibration step, different concentrations of creatinine standard were spiked into each individual sample. The concentration ranged from 0 mg/dL to 11.3 mg/dL, which is the typical range of creatinine in the human body. The concentration of the creatinine in each sample was calculated according to its specific calibration curve. The total detection time from sample loading was <5 min. The sample volume requirement was 40 μL.

### Calibration Curve for Creatinine Standards

In order to get an accurate readout of the creatinine concentration, calibration curves were obtained for each individual sample. In the calibration experiment, creatinine standards were spiked into the blood samples at different concentrations from 0 mg/dL to 11.3 mg/dL by serials dilution, which covers the dynamic range of blood creatinine level. For each dilution, the dilution ratio is 1:1.5 from 11.3 mg/dL. The signals result in a linear relationship to the spiked creatinine concentration. By a linear regression fitting process, the accurate concentration of the blood sample was interpreted according to this calibration curve (FIG. 2D). The linear fitting equation is ΔI (nA)=98.98+19.17×creatinine concentration (mg/dL). The limit of detection (LOD) for the electrochemical sensor was calculated based on the two standard deviation (SDV) cutoff from the calibration curves. According to the calibration curve, the LOD for creatinine was 0.46 mg/dL. The overall estimation of all the calibration curves from the 19 samples demonstrates very similar fitting parameters. The average slope is 19.35±1.58. Alternatively, a single universal calibration curve may be generated that simplifies the process for clinical application.

### Clinical Samples Measurement

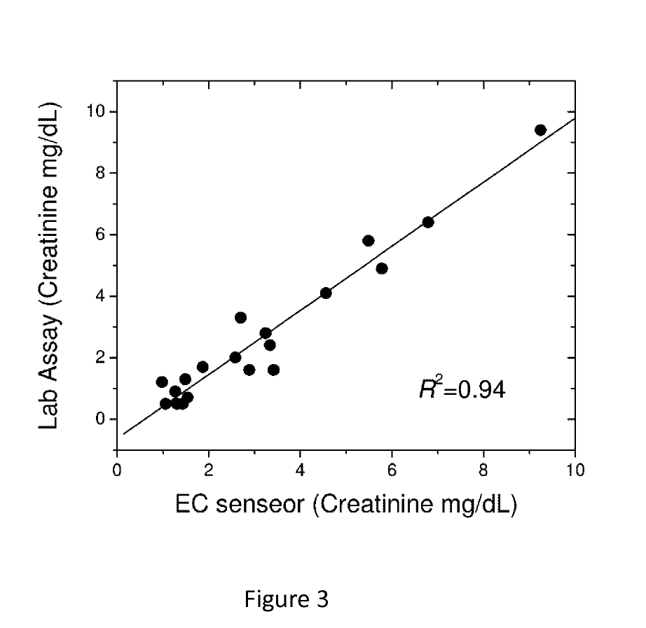

Clinical blood samples were tested with the electrochemical sensor. For each sample, 40 μL volume was utilized during the test. Ten microliters (10 μL) of HRP-conjugated anticreatinine antibody was first mixed with 40 μL of raw blood sample solution at room temperature. The mixture then was transferred onto the electrodes for competitive reaction between the creatinine in solution and on the polymer matrix. Each measurement took <5 min from the sample loading by pipetting the mixture of HRP-conjugated antibody and blood sample onto the electrode. For comparison, the creatinine concentration was also measured using the traditional Jaffe methodology at the UCLA Ronald Reagan Clinical Laboratory. FIG. 3 lists the creatinine concentration from the 19 clinical samples based on electrochemical sensor and the Jaffe reaction. The linear correlation between the two methods is also presented in FIG. 3. The EC sensor has very comparable sensitivity with the traditional Jaffe reaction (the correlation coefficient, R², is 0.94). Of note, the EC sensor required only 40 μL of blood sample, compared to the 1000 μL required by the Jaffe reaction. In addition, the measurement based on the EC sensor took <5 min to complete, compared to over 1 h for the Jaffe reaction.

Point-of-care testing (POCT) testing is defined as medical testing at the site of patient care. The glucometer-POCT device has revolutionized the quality of life and insulin regulation for diabetic patients. The rapid and portable detection of creatinine would have a tremendous impact on the quality of life for renal transplant recipients, while enabling rejection to be determined at an earlier stage, when it is more easily reversed. This technology could be potentially applied to oncology and radiology specialties, where the rapid detection of creatinine is important prior to the induction of chemotherapy and to avoid contrast-induced nephropathy. Four promising POCT devices to measure whole blood creatinine have already been brought to the marketplace, namely IRMA TRUpoint (ITC, Edison, N.J.), Radiometer ABL800 FLEX (Radiometer A/S, Bronshoj, Denmark), StatSensor (Nova Biomedical, Waltham, Mass.), and i-STAT (Abbott Diagnostics, East Windsor, N.J.). While TRUpoint and Radiometer are larger multifunctional benchtop analyzers, StatSensor and i-STAT (crea-cartridge) are hand-held POCT devices that can rapidly measure whole blood creatinine Both i-STAT and StatSensor utilize similar enzymatic chemical reactions to detect creatinine electrochemically (Korpi-Steiner et al., 2009 Am J Clin Pathol 132:920-926; Gault et al., 2001, Nephron 88:178-182). The i-Stat device has been available for almost a decade and has seen some slow adoption by radiology and emergency departments. Detractors of this device report consistent overestimation of creatinine, and the 100-μL volume requirement makes it improbable for fingerprick sampling (Korpi-Steiner et al., 2009 Am J Clin Pathol 132:920-926; Gault et al., 2001, Nephron 88:178-182; Nichols et al., 2007, Clin Chim Acta 377:201-205). StatSensor is a new device with considerable potential, because it is simple to use and can measure creatinine from a fingerprick sample. Unfortunately at least 2 previous studies have reported that the StatSensor did not meet expectations, was imprecise and consistently underestimated creatinine levels (Korpi-Steiner et al., 2009 Am J Clin Pathol 132:920-926; Shephard et al., 2010, Clin Chem Lab Med 48:1113-1119). Shephard et al. concluded that the StatSensor needs urgent improvement (Shephard et al., 2010, Clin Chem Lab Med 48:1113-1119).

Usually, with the enzymatic procedure, multiple catalysis steps are required, with the final step being the measurement of small chemicals generated from the previous reaction. Interference with analogues found in body fluids, such as glucose, fructose, ketone bodies, ascorbic acid, and cephalosporins (Benkert et al., 2000, Electroanalysis 12:1318-1321; Lo and Tsai, 1994, Clin Chem 40:2326-2327) may affect the accuracy of this final measurement. By using an antibody-mediated amperometric system rather than an enzymatic-mediated amperometric system, it is anticipated that the interfering signals faced by these other devices when working with a complex matrix such as whole blood (Benkert et al., 2000, Anal Chem 72:916-921) are avoided. This is especially important to renal transplant recipients, who often have hyperglycemia and are taking multiple immunosuppressant medications that could act as confounders.

The creatinine EC sensor in this study provides a rapid and accurate way for serum creatinine measurement. By applying the cyclic square wave during the measurement, the detection time was <5 min from the sample loading, compared with other electrochemical sensor based on passive reaction. The procedure of EC creatinine sensor requires simply mixing the HRP-creatinine antibody with the target sample before loading to the electrodes. Sensitivity of the detection was found to be 0.46 mg/dL of creatinine with only 40 μL sample in the creatinine concentration range of 0 mg/dL to 11.3 mg/dL. This is the ideal clinical range for detecting graft dysfunction.

However, converting the POCT device for immediate clinical application would be limited by sample volume fluctuations. Standardization in the sample-processing step is necessary for different sample volumes affect the signal readout of the device. Therefore, an additional sample-processing accessory would need to be developed, so that 40 μL of blood can consistently be delivered to the sensor, in a manner that is “user-friendly” for the patient.

This rapid assay for creatinine detection by point-of-care testing (POCT) was able to produce consistent signal levels that accurately detected the creatinine concentrations from clinical blood samples. The creatinine sensor covered the desired clinical range for detecting allograft dysfunction (0 mg/dL to 11.3 mg/dL). Signal levels that were detected electrochemically correlated closely with the creatinine blood concentrations reported by the Ronal Reagan UCLA Medical Center traditional clinical laboratory assay (R²=0.94). With the development of an accessory that could consistently deliver 40 μL of blood to the sensor, this device could become a prominent clinical diagnostic tool for measuring allograft dysfunction in renal transplantation.

### Example 2

### Multiplexing ESensor

The multiplexing electrochemical sensor is based on the simultaneous detection of different types of renal function markers. In this example, the combination of creatinine and cystatin-C is provided. Creatinine is a small chemical, and the detection is a competitive amperometric measurement. Cystatin-C detection is performed via a sandwich protein detection assay, as shown schematically in FIG. 4.

### Multiplexing Measurements for Different Types of Molecules

Conducting polymer (CP) has been applied in this rapid and simple procedure. By applying the co-polymerization with polymer matrix, different types of capture molecules can be directly embedded simultaneously onto the surface of the same chip. The total reaction time is from several seconds to minutes, at room temperature with regular biocompatible buffer.

A three-electrode system has been utilized in this assay. Each unit of the array has a working electrode, a counter electrode, and a reference electrode. The 3-electrodes are bare gold before the reaction, and the specimens are immobilized on the working electrode. Electrochemical current is measured between the working electrode and counter electrode under the potential between the working electrode and the reference electrode. The potential profile could be a constant value, a linear sweep or a cyclic square wave. For the experiments, solutions were loaded onto the whole area of the three-electrode region including the working, counter, and reference electrodes.

### Conducting Polymer Sensor Fabrication:

A cyclic square-wave electrical field (CSW E-field) was applied for the electropolymerization and surface-recognition processes, which provides more effective and versatile way to control the assay. With the CSW E-field, both the reaction and protein binding are finished on the same chip within minutes, while previously these processes have to be completed separately and the incubation time varies from 1-24 hours. The positive potential in the CSW E-field help to accumulate the molecules onto the working electrode, while the negative potential removes the weak non-specific binding which generate high specificity. The flapping between positive and negative potential also provides good mixing during the incubation, which accelerates the binding process as well. Each cycle of square-wave consisted of about 9 s at low voltage and about 1 s at high voltage. For EC polymerization, the low voltage was about +350 mV and the high voltage was about +950 mV. For the surface recognition, the low voltage was about −200 mV and the high voltage was about +300 mV. In total, about 20 cycles of square-waves were applied for each surface reaction, which lasted for about 200 s. The parameters during the electro-polymerization need to be optimized depending on the specific molecules, including the voltage, time duration, and cycle numbers. For example, creatinine polymerization is carried out as the following: The gold electrochemical sensor is first coated with creatinine embedded in the polyrrole conducting polymer. For electropolymerization, the 20 mg/dL creatinine (Abnova, USA) was diluted together with pyrrole (Sigma, USA) in 1×PBS (pH 7.5, Invitrogen, USA) in a volume ratio of 1:50. Potassium chloride was added at a final concentration of 300 mM to achieve high ionic strength. The final concentration of pyrrole was 10 mM. After loading of the mixture onto the gold electrode, a CSW E-field was applied for electropolymerization. Each square-wave consisted of 9 s at a potential of +350 mV and 1 s at +950 mV, and 20 cycles of square-waves were applied. The whole process lasted for 200 s. After the polymerization, the electrode was rinsed with ultrapure water (18.3 MΩ·cm) then dried under pure N₂. Polymerization was performed similarly for creation of the cystatin-C chip, however an anti-cystatin-C antibody was mixed with pyrrole and electropolymerized to functionalize the surface of the working electrode chip.

### Amperometric Measurement

The final concentration is reported based on the individual calibration curve for each sample. In the multiplexing reading, different reporting molecules are required. For creatinine, the antibody is a sheep polyclonal antibody against creatinine (Abnova, USA), there is also an antibody against cystatin-C. HRP (Horseradish peroxidase) was conjugated to the antibody with the NH₂-biotin-label kit, following the enclosed instruction from the company (Dojindo, USA).

10 μl of HRP conjugated antibody was first mixed with 4 μl raw blood sample solution at room temperature. Then the mixture was transferred onto the electrodes for reaction. 20 cycles of square-wave consisted of 9 s at a potential of −300 mV and 1 s at +200 mV were applied. The whole process lasted for 200 s. After that, the sensor was washed with water and then dried under pure N₂. Then the amperometric measurements were carried out in the presence of 3,3-,5,5-tetramethylbenzidine (TMB/H₂O₂, Neogen Corp., USA) low-activity substrate at −200 mV. The decreased current of the HRP-antibody is proportional to the level of creatinine in the samples. The electrochemical signal was the current generated by the redox cycles between TMB, the HRP reporter enzyme, and H₂O₂. All experiments were performed at room temperature.

### Sensitivity and Specificity

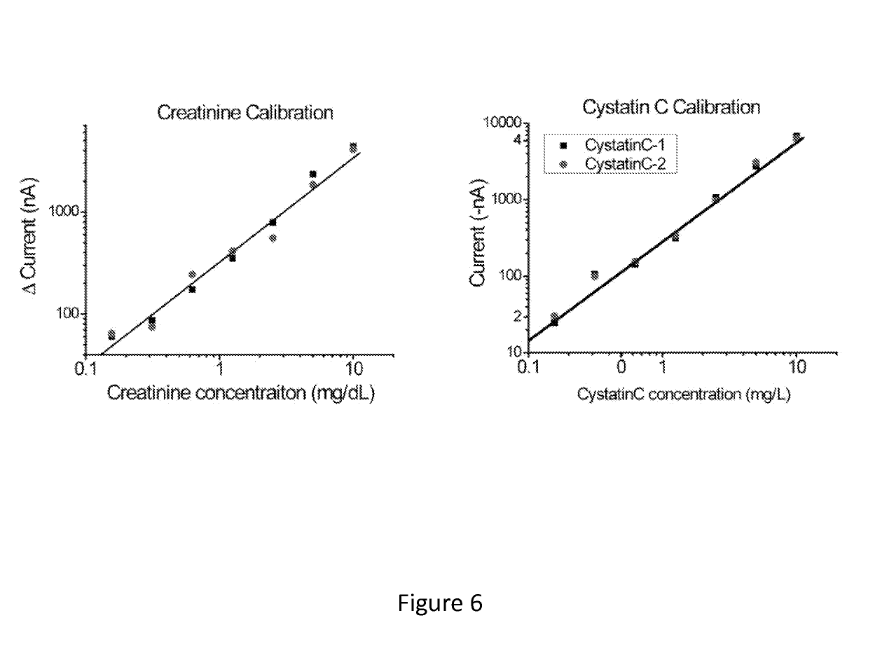

The assay was validated in both buffer and spiked blood samples. For the buffer system, the limit-of-detection (LOD) is 0.2 ug/dL for creatinine and 1 pg/ml for cystatin-C. The dynamic range is 20 mg/dl to 0.2 ug/dl for creatinine and 1 ug/ml to 1 pg/ml for cystatin-C, as shown in FIG. 5. Validation has also been carried out in spiked blood samples. 195 clinical blood samples were pooled. Then standards of creatinine and cystatin-C were spiked into the pooled blood samples by serial dilution, as shown in FIG. 6.

### Robustness

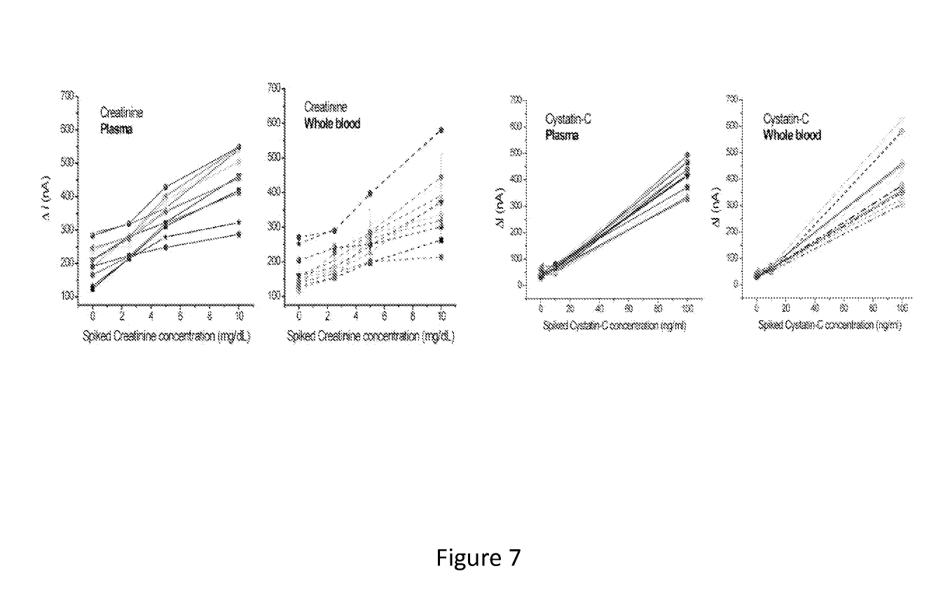

The robustness of the assay has been investigated in 10 blood samples. For each individual sample, creatinine and cystatin-C are measured simultaneously. Calibration curves in pooled samples have similar slopes. Pooled samples have lower standard deviations. Sample types are also studied by comparing the results from whole blood and plasma. Plasma has lower variation between different samples, as shown in FIG. 7.

### Clinical Test

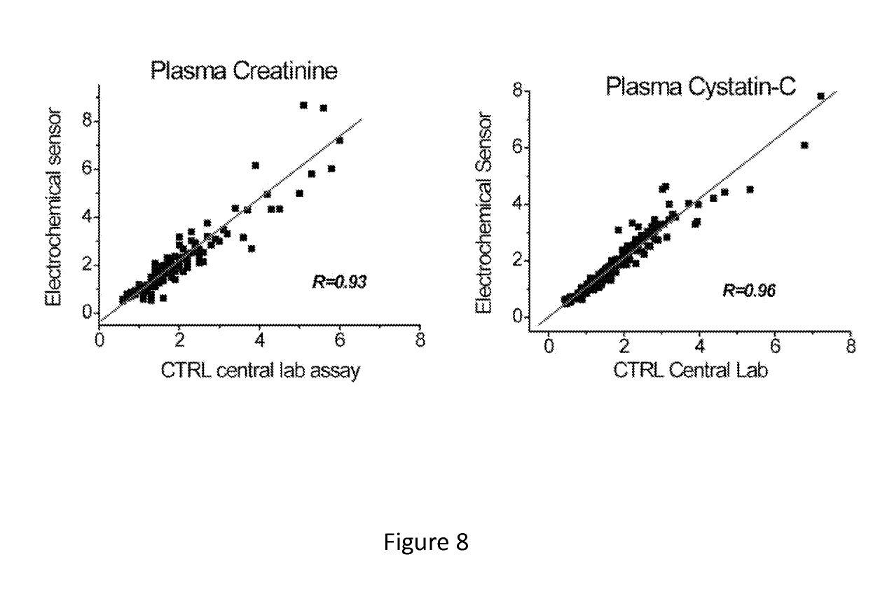

200 clinical plasma samples from renal transplant recipients were tested by the assay, including rejection patients, stable patients, and healthy controls. For each individual subject, blood was drawn at three different stages: 1—before biopsy; 2—during biopsy; and 3—after biopsy. First, the comparison between the multiplexing assay of the present invention and the traditional lab assay are illustrated. For the plasma creatinine assay, the linear fitting has a correlation coefficient of R=0.9923. For the plasma cystatin-C assay, the linear fitting has a correlation coefficient of R=0.9944, as shown in FIG. 8.

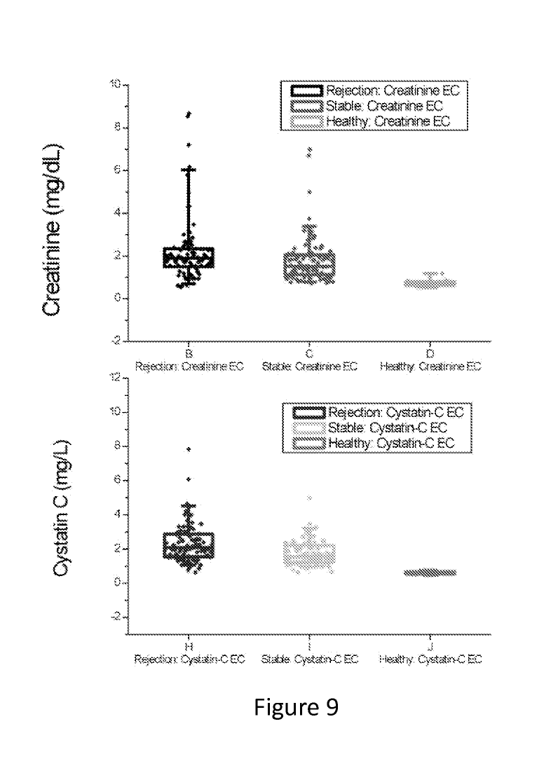

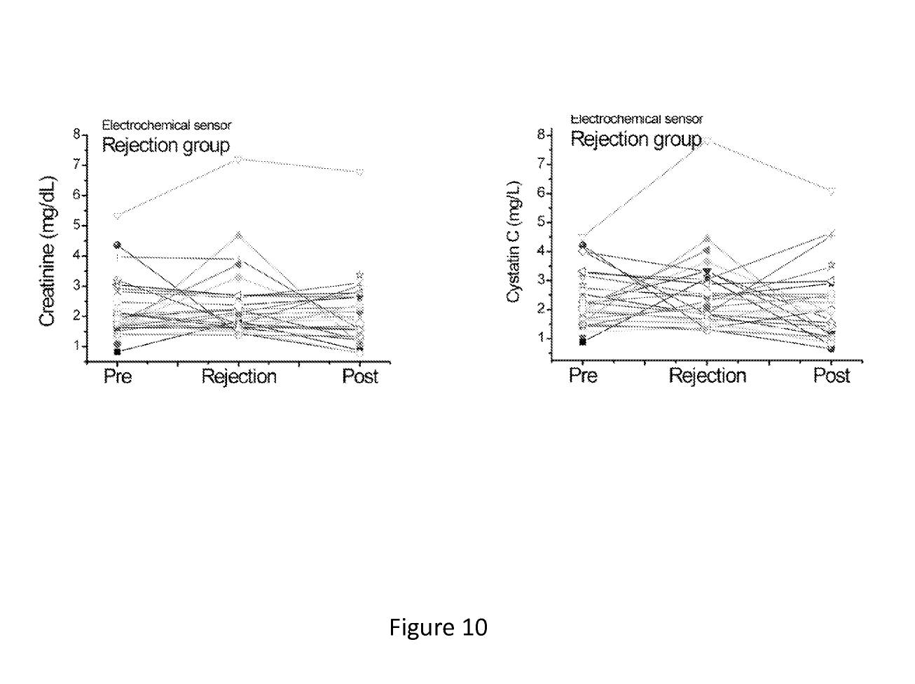

The assay of the present invention also demonstrates that the system can differentiate between the rejection, stable and health controls, as shown in FIG. 9. Further, the present invention also demonstrates the potential to monitor patients while they transition through the dynamic stages of rejection, as shown in FIG. 10.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A device for monitoring renal function of a subject, comprising:
an array of units on a substrate, each unit comprising an electrode chip including a working electrode, a counter electrode, and a reference electrode;
wherein the working electrode of at least one unit is coated with a conducting polymer embedded or functionalized with a first marker of renal function.

2. The device of claim 1, wherein the working electrode, counter electrode, and reference electrode are comprised of a conductive material.

3. The device of claim 1, wherein the first marker of renal function is creatinine.

4. The device of claim 1, wherein the current in the electrode chip is altered when the first marker of renal function binds to an antibody directed against the first marker.

5. The device of claim 4, wherein the antibody is conjugated to horseradish-peroxidase (HRP).

6. The device of claim 1, wherein the conducting polymer comprises pyrrole.

7. The device of claim 1, wherein the conducting polymer is electropolymerized on the working electrode by applying a cyclic square-wave electric field to the device.

8. The device of claim 1, wherein the working electrode of at least one unit is coated with a conducting polymer embedded with an antibody directed against a second marker of renal function.

9. The device of claim 8, wherein the current in the electrode chip is altered when the antibody directed against the second marker binds to the second marker.

10. The device of claim 8, wherein the second marker of renal function is cystatin-C.

11. A method of monitoring renal function in a subject comprising:
obtaining a blood sample from the subject;
mixing a first portion of the sample with a solution comprising an antibody directed against a first marker of renal function to form a mixture;
adding the mixture to a first electrode chip on a device, the electrode chip comprising a working electrode, a counter electrode, and a reference electrode; wherein the working electrode is coated with a conducting polymer embedded with the first marker of renal function; and
measuring the current in the electrode chip, wherein the magnitude of a change in current, compared to the current measured when a mixture comprising the antibody but not comprising the sample is added to the electrode chip, is correlated to the quantity of the first marker in the sample.

12. The method of claim 11, wherein the first marker of renal function is creatinine.

13. The method of claim 11, further comprising applying a cyclic square-wave electric field to the electrode chip during the adding step.

14. The method of claim 11, further comprising:
Mixing a second portion of the sample with a solution comprising an antibody directed against a second marker of renal function to form a second mixture;
adding the second mixture to a second electrode chip on the device, the second electrode chip comprising a working electrode, a counter electrode, and a reference electrode; wherein the working electrode is coated with a conducting polymer embedded with an antibody directed against the second marker of renal function;
measuring the current in the second electrode chip, wherein the magnitude of the current is correlated to the quantity of the second marker in the sample.

15. The method of claim 14, wherein the second marker of renal function is cystatin-C.

16. A system for monitoring renal function in a subject, comprising:
an electrochemical sensor chip having at least one well, wherein the at least one well contains a working electrode coated with a conducting polymer functionalized with at least one capture molecule; and
at least one antibody that specifically binds to a biomarker of renal function;
wherein the at least one antibody is mixed with a blood sample from the subject and added to the at least one well, such that when an electric current is applied to the sample in the at least one well, at least some the biomarker binds to the capture molecule, thereby creating a measurable change in electric current in the sample that is indicative of the presence of the biomarker in the sample.

17. The system of claim 16, wherein the biomarker is creatinine.

18. The system of claim 17, wherein the capture molecule is creatinine.

19. The system of claim 16, further comprising:
a first antibody that specifically binds to creatinine and a second antibody that specifically binds to cystatin-C; and
at least two wells in the electrochemical sensor chip, wherein the capture molecule in the first well specifically binds to creatinine and the capture molecule in the second well specifically binds to cystatin-C.

20. The system of claim 16, wherein the change in current in the sample is measurable within 5 minutes after the sample has been loaded into the well.
